(19) United States
(12) Patent Application Publication
(10) Pub. No.: US 2020/0117830 A1
(43) Pub. Date: Apr. 16, 2020

(54) INFORMATION PROCESSING SYSTEM AND INFORMATION PROCESSING APPARATUS

(71) Applicant: CANON KABUSHIKI KAISHA, Tokyo (JP)

(72) Inventor: Toshimizu Yamane, Tokyo (JP)

(21) Appl. No.: 16/713,371

(22) Filed: Dec. 13, 2019

Related U.S. Application Data
(63) Continuation of application No. PCT/JP2018/022285, filed on Jun. 11, 2018.

(30) Foreign Application Priority Data
Jun. 15, 2017 (JP) 2017-117681

(51) Int. Cl. G06F 21/62; G16H 10/60; G16H 40/20; G16H 30/20

(52) U.S. Cl. CPC G06F 21/6245; G16H 30/20; G16H 40/20; G16H 10/60

(57) ABSTRACT

Provided is an information processing system including: first and second information processing apparatuses. The first information processing apparatus includes: a first storage unit storing personal information of a patient, medical information, and identification information in association with one another; and a first update unit updating the identification information stored in the first storage unit. The second information processing apparatus includes: a second storage unit storing the medical information and the identification information in association with each other; an associating unit associating the personal information received by a reception unit with one piece of identification information; and a second update unit updating the identification information stored in the second storage unit. The first and second update units update a first piece of identification information associated with the same medical information in the first and second storage units, respectively at corresponding timings, to a second piece of identification information.

### CROSS-REFERENCE TO RELATED APPLICATIONS

This application is a Continuation of International Patent Application No. PCT/JP2018/022285, filed Jun. 11, 2018, which claims the benefit of Japanese Patent Application No. 2017-117681, filed Jun. 15, 2017, both of which are hereby incorporated by reference herein in their entirety.

### BACKGROUND

### Field

The present disclosure relates to an information processing system and an information processing apparatus.

### Description of the Related Art

In recent years, image processing and other such medical information services for examination images and other such images obtained in a hospital have been increasingly performed in a data center outside the hospital, for example, a cloud. In such services, from the viewpoint of protecting personal information of each patient, it is required to remove the personal information from a medical image being diagnostic information on a gateway server in the hospital and upload only the medical image to the data center. The data center is configured to return an identifier for an uploaded image so as to allow the gateway server to identify, operate, and refer to the uploaded image. This identifier is hereinafter referred to as “association ID”. The gateway server manages the patient information and the association ID of the image in association with each other in order to associate the patient information with a result of the image processing, which is returned from the data center with respect to the uploaded image. In Japanese Patent Application Laid-Open No. 2010-264107, there is disclosed a technology in which identification information unique to a medical image being diagnostic information is used as an association ID when the medical image is uploaded to a data center outside a hospital.

In recent years, there has been an increase in the importance of secondary use of data, for example, use of a large amount of big data including medical images accumulated in a data center for the purpose of performing statistical processing and creating a case image database. For secondary use of the diagnostic information, a certain amount of personal information including the age and the gender of each patient is required. However, there is a high risk of information leakage from a data center outside a hospital, and hence it is required to take into consideration the protection of personal information.

However, in the technology disclosed in Japanese Patent Application Laid-Open No. 2010-264107, each medical image becomes anonymized data identified only by the association ID in the data center. This inhibits the medical image from being secondarily used. There is also a problem in that, in a case where information unique to the medical image is used as the association ID, when the information has been leaked, the personal information leaked in the past can be connected to the medical image, which increases the possibility that an individual can be identified by the information.

The present disclosure has been made in view of such problems, and can provide a mechanism capable of secondary use of a medical image of a patient while protecting personal information of the patient.

### SUMMARY

According to at least one embodiment of the present disclosure, there is provided is an information processing system including: a first information processing apparatus; and a second information processing apparatus, the first information processing apparatus including: a first storage unit configured to store personal information of a patient, medical information obtained for a person relating to the personal information, and identification information for identifying the medical information in association with one another; and a first update unit configured to update the identification information stored in the first storage unit, the second information processing apparatus including: a second storage unit configured to store the medical information and the identification information in association with each other; a reception unit configured to receive at least a part of the personal information associated with one piece of identification information from the first information processing apparatus; an associating unit configured to associate the personal information received by the reception unit with the one piece of identification information; and a second update unit configured to update the identification information stored in the second storage unit, and the first update unit and the second update unit configured to update a first piece of identification information associated with the same medical information in the first storage unit and the second storage unit, respectively at corresponding timings, to a second piece of identification information different from the first piece of identification information.

Further features of the present disclosure will become apparent from the following description of exemplary embodiments with reference to the attached drawings.

### BRIEF DESCRIPTION OF THE DRAWINGS

FIG. 1 is an overall diagram of a medical system in a first embodiment of the present disclosure.

FIG. 2 is a hardware configuration diagram of a client apparatus.

FIG. 3 is a sequence diagram for illustrating upload processing to be performed by the medical system.

FIG. 4 is a diagram for illustrating an example of data structures of data storage areas after the upload processing.

FIG. 5 is a sequence diagram for illustrating job request processing to be performed by the medical system.

FIG. 6 is a diagram for illustrating an example of a data structure of a request message.

FIG. 7 is a diagram for illustrating an example of data structures of the data storage areas.

FIG. 8 is a sequence diagram for illustrating secondary use processing to be performed by the medical system.

FIG. 9 is a diagram for illustrating an example of data structures of the data storage areas.

FIG. 10 is a diagram for illustrating an example of data structures of the data storage areas.

FIG. 11 is a diagram for illustrating an example of data structures of the data storage areas.

FIG. 12 is an explanatory diagram of secondary use data.

FIG. 13 is a flow chart for illustrating update control processing for an association ID.

FIG. 14 is a diagram for illustrating an example of data structures of the data storage areas.

FIG. 15 is a diagram for illustrating an example of data structures of the data storage areas.

FIG. 16 is a sequence diagram for illustrating secondary use processing.

FIG. 17 is a diagram for illustrating an example of data structures of the data storage areas.

### DESCRIPTION OF THE EMBODIMENTS

In the following description, like components are denoted by like reference symbols. Preferred embodiments of the present disclosure will now be described in detail in accordance with the accompanying drawings.

### First Embodiment

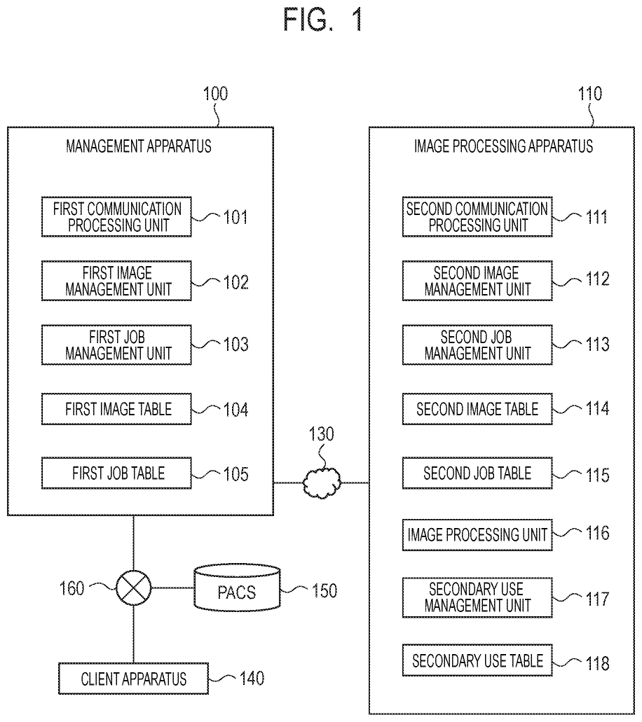

FIG. 1 is an overall diagram of a medical system in a first embodiment of the present disclosure. The medical system is an example of an information processing system. The medical system includes a management apparatus 100 being an example of an information processing apparatus, and an image processing apparatus 110 being another example of the information processing apparatus. The management apparatus 100 is set in a hospital, and manages examination images and other such medical images of patients together with personal information of the patients. The medical image is hereinafter referred to simply as “image”. The management apparatus 100 is, for example, a gateway server. The image processing apparatus 110 is an image processing server placed on a cloud, and performs image processing on the image received from the management apparatus 100 to return a result of the processing to the management apparatus 100. In the first embodiment, subtraction image generation is employed as an example of the image processing, but fusion image generation or other such image processing may be employed. The management apparatus 100 and the image processing apparatus 110 are connected to each other through an Internet 130. A client apparatus 140 is also an example of the information processing apparatus, and is used by a doctor or another such user to display and operate the image. The user accesses the management apparatus 100 through use of the client apparatus 140, to thereby be able to request the image processing apparatus 110 to perform image processing, and display and operate a result of the image processing. In addition, a picture archiving and communication system (PACS) 150 is an apparatus for storing images. The client apparatus 140, the PACS 150, and the management apparatus 100 are connected to one another through an intranet 160.

The management apparatus 100 includes, as its functional components, a first communication processing unit 101, a first image management unit 102, a first job management unit 103, a first image table 104, and a first job table 105. The first communication processing unit 101 controls communication to/from an external apparatus. The first image management unit 102 is an example of an update unit, an update control unit, and a management unit, and manages an image stored in the own apparatus. The first job management unit 103 manages a job ID of a job of the image processing for the image to be executed by the image processing apparatus 110. The first image table 104 is an example of a storage unit, and stores an image and the personal information of the patient corresponding to the image in association with each other. The first job table 105 stores a job ID assigned to image processing in the image processing apparatus 110 and an image relating to the image processing (job) in association with each other. In this case, the job ID represents the identification information of the job.

The image processing apparatus 110 includes, as its functional components, a second communication processing unit 111, a second image management unit 112, a second job management unit 113, a second image table 114, and a second job table 115. The image processing apparatus 110 further includes an image processing unit 116, a secondary use management unit 117, and a secondary use table 118. The second communication processing unit 111 is an example of a reception unit and a transmission unit, and controls communication to/from an external apparatus. The second image management unit 112 is an example of an update unit, an update control unit, an issuance unit, and a management unit, and receives an image from the management apparatus 100, and manages an image stored in the own apparatus. The second job management unit 113 manages a job of image processing for an image. The second image table 114 is an example of a storage unit, and stores an image. The second job table 115 is an example of a third storage unit, and stores a job ID and an image in association with each other.

The image processing unit 116 is an example of a processing unit, and performs image processing on an image. The secondary use management unit 117 is an example of an associating unit, and manages secondary use of an image. In this case, examples of the secondary use include using an image, which was received from the management apparatus 100 originally for the purpose of being subjected to image processing by the image processing unit 116, for statistical processing by comparing the image with the personal information of a person corresponding to the image. Processing relating to the secondary use may be performed by the image processing apparatus 110 or another apparatus in the same cloud. When an image is secondarily used in another apparatus, the image processing apparatus 110 transmits the image to be secondarily used to another apparatus through use of a communication channel closed in the cloud together with required personal information. The secondary use table 118 stores information relating to the secondary use.

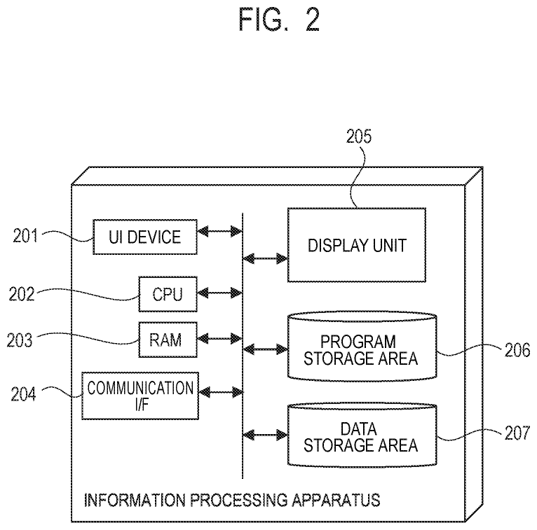

FIG. 2 is a hardware configuration diagram of the client apparatus 140. A UI device 201 is, for example, a mouse, a keyboard, or a touch sensor configured to detect a position touched by, for example, a finger on a display screen. An instruction issued from the user is input to the UI device 201. A CPU 202 reads and loads a program from a program storage area 206 into a RAM 203, and interprets and executes the program, to thereby implement each of various kinds of control, calculation, UI display, and other such operations. A communication I/F 204 is connected to the intranet 160 and transmits and receives data to/from the management apparatus 100, the image processing apparatus 110, and other such external apparatus. A display unit 205 displays states of the apparatus and details of processing. The display unit 205 is, for example, a light emitting diode (LED) or a liquid crystal panel. Various programs are stored in the program storage area 206. Various kinds of data are stored in a data storage area 207. The program storage area 206 and the data storage area 207 are formed of, for example, a hard disk drive or a flash memory, but the storage medium to be employed is not limited thereto.

The hardware configuration of the management apparatus 100 is the same as the hardware configuration of the client apparatus 140 described with reference to FIG. 2. However, the management apparatus 100 is not required to include the UI device 201 and the display unit 205. The communication I/F 204 of the management apparatus 100 is also connected to both the intranet 160 and the Internet 130. The functions of the management apparatus 100 described with reference to FIG. 1 and the processing described later, for example, are implemented by the CPU 202 of the management apparatus 100 reading a program and executing the program.

The hardware configuration of the image processing apparatus 110 is the same as the hardware configuration of the management apparatus 100. However, the communication I/F 204 of the image processing apparatus 110 is connected to the Internet 130, but is not connected to the intranet 160. The functions of the image processing apparatus 110 and the processing described later are implemented by the CPU 202 of the image processing apparatus 110 reading a program and executing the program. The first image table 104 and the first job table 105 are assumed to be stored in the data storage area 207 of the management apparatus 100. It is also assumed that the second image table 114, the second job table 115, and the secondary use table 118 are stored in the data storage area 207 of the image processing apparatus 110.

As another example, the management apparatus 100 includes a plurality of CPUs, ROMs, and RAMs, and each of the functions described with reference to FIG. 1 and the processing described later may be implemented by the plurality of CPUs, ROMs, RAMs, and other such components in cooperation with one another. As still another example, each of the functions and processing of the management apparatus 100 may be implemented by a hardware circuit. The same applies to the image processing apparatus 110.

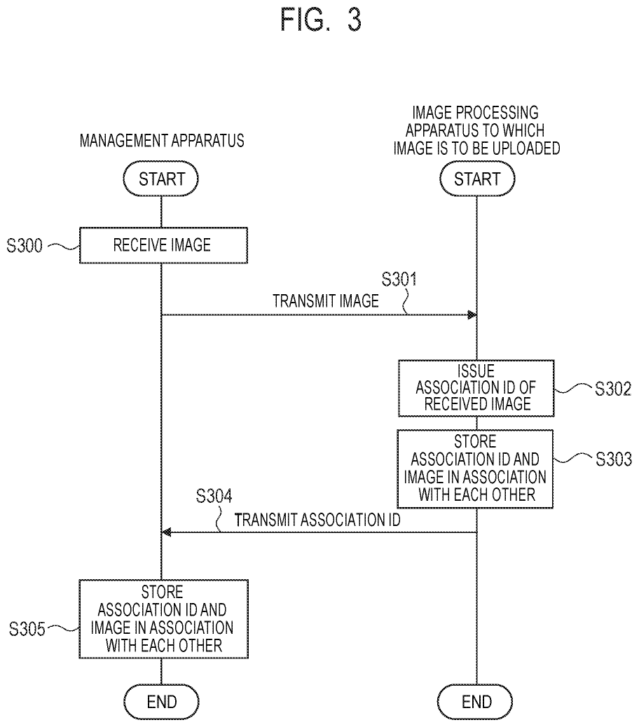

FIG. 3 is a sequence diagram for illustrating upload processing to be performed by the medical system. The upload processing is processing for transmitting an image from the management apparatus 100 to the image processing apparatus 110 so that the image processing apparatus 110 performs image processing on the image stored in the management apparatus 100. In Step S300, the first image management unit 102 receives, from the PACS 150, an image designated by the user. Specifically, when the client apparatus 140 transmits information for designating the image selected by a user operation to the management apparatus 100, the first image management unit 102 acquires the image from the PACS 150 through the first communication processing unit 101 based on the information for the designation. Subsequently, in Step S301, the first communication processing unit 101 transmits the image acquired in Step S300 to the image processing apparatus 110. At this time, the first communication processing unit 101 avoids transmitting personal information (for example, patient name, gender, and age) included as meta information for the image.

When the second communication processing unit 111 of the image processing apparatus 110 receives the image in Step S301, the second image management unit 112 then issues an association ID for the received image in Step S302. In this case, the association ID is an example of identification information for identifying the image. In addition, this processing to be performed by the second image management unit 112 is an example of issuance processing for issuing the identification information of the image. Subsequently, in Step S303, the second image management unit 112 stores the image received in Step S301 and the association ID issued in Step S302 in the second image table 114 in association with each other. This processing to be performed by the second image management unit 112 is an example of management processing for the second image table 114. In this case, the second image table 114 may store a part of the personal information together with the image. Subsequently, in Step S304, the second communication processing unit 111 transmits the association ID issued in Step S302 to the management apparatus 100. When the first communication processing unit 101 of the management apparatus 100 receives the association ID in Step S304, the first image management unit 102 then stores, in Step S305, the association ID, the image, and the personal information included as meta information for the image in the first image table 104 in association with one another. This processing to be performed by the first image management unit 102 is an example of management processing for the first image table 104.

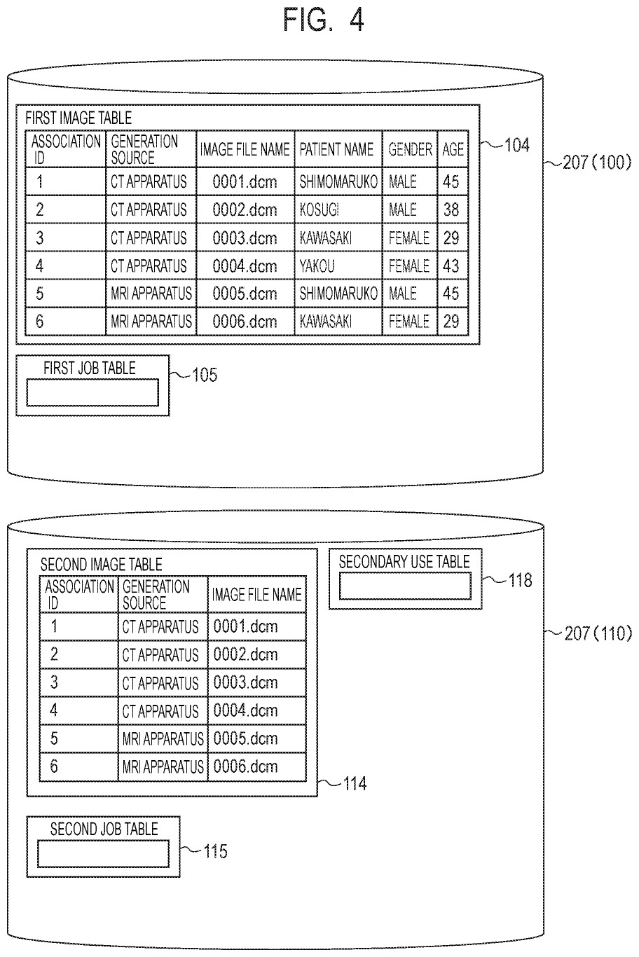

FIG. 4 is a diagram for illustrating an example of data structures of the data storage areas 207 after the upload processing. The first image table 104 of the management apparatus 100 stores a record (row of FIG. 4) corresponding to each of a plurality of uploaded images. In the example of FIG. 4, six records corresponding to six images are stored. Each record includes an association ID, a generation source, an image file name, a patient name, a gender, and an age. In this case, the patient name, the gender, and the age are information on the patient, and are examples of information included in the personal information. The number of pieces of information and the kinds of information included in the personal information are not limited to those in the first embodiment, and may be freely determined. The generation source is information indicating an apparatus that has generated the image. The data storage area 207 of the management apparatus 100 also stores main image data in association with the image file name.

In addition, the second image table 114 of the image processing apparatus 110 stores records corresponding to each of the plurality of uploaded images. The first image table 104 and the second image table 114 store the records corresponding to the same image. Each of the records includes the association ID, the generation source, and the image file name. The data storage area 207 of the image processing apparatus 110 stores the image itself in association with the image file name. The personal information is not stored in the second image table 114. At this time point, no records (data) are stored in the first job table 105, the second job table 115, and the secondary use table 118.

As another example, when transmitting (uploading) an image, the management apparatus 100 may transmit not only the image but also a part of less confidential information or other such personal information. In this case, the part of the personal information is stored in the second image table 114 in association with the image.

Through the upload processing described with reference to FIG. 3, the first image table 104 and the second image table 114 store the same image ID in association with the same image. This allows the management apparatus 100 and the image processing apparatus 110 to determine the identity of the images stored in the respective apparatus based on the association ID.

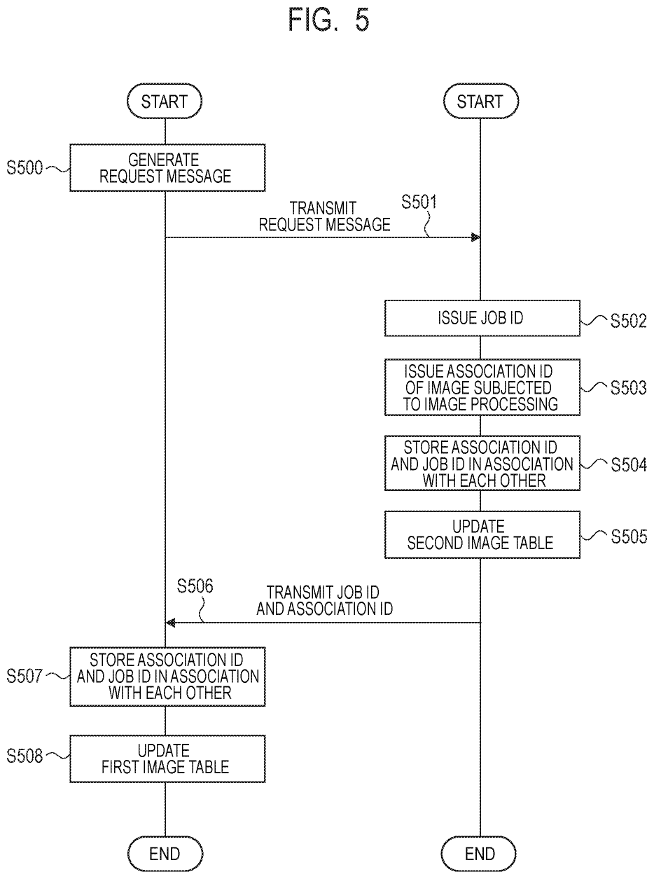

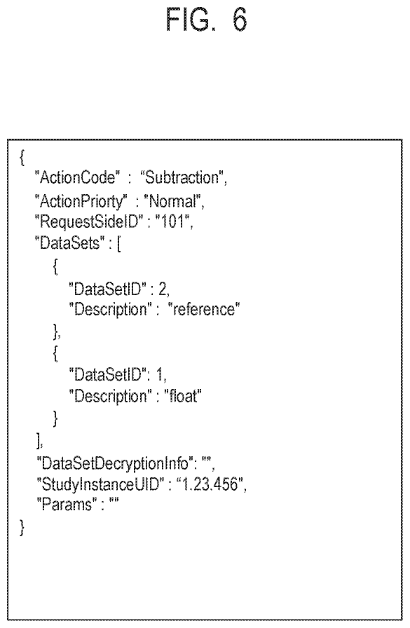

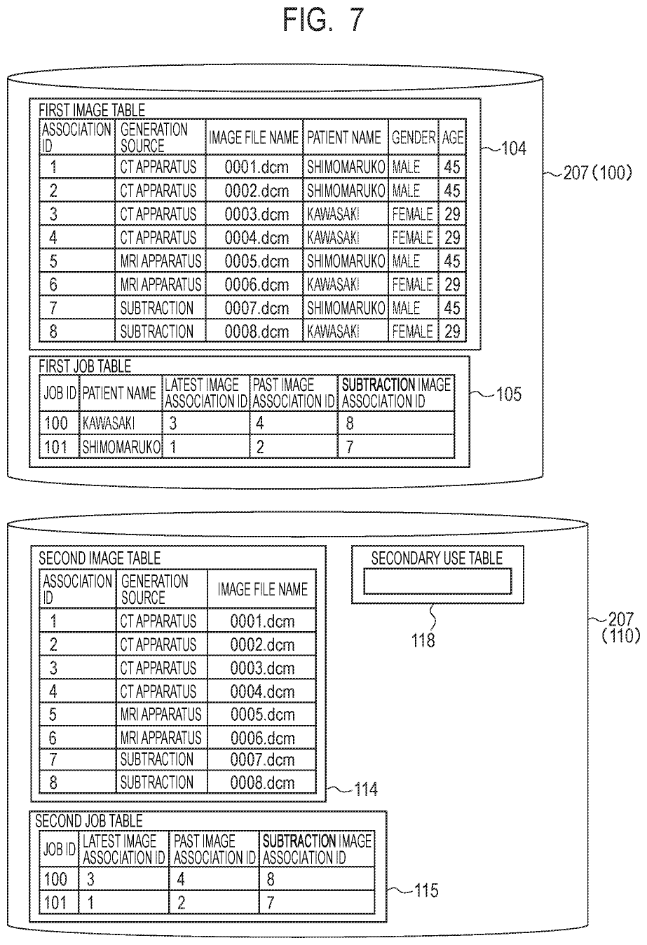

FIG. 5 is a sequence diagram for illustrating job request processing to be performed by the medical system. The job request processing is processing for transmitting a request for an image processing job from the management apparatus 100 to the image processing apparatus 110 so that the image processing apparatus 110 performs the image processing on the uploaded image. FIG. 6 is a diagram for illustrating an example of a data structure of a request message to be transmitted from the management apparatus 100 in the job request processing. FIG. 7 is a diagram for illustrating an example of data structures of the data storage areas 207 after the job request processing. In the first embodiment, the job request processing is described by taking an exemplary case in which image processing relating to a request is processing for generating a subtraction image between two images.

In Step S500 illustrated in FIG. 5, the first job management unit 103 acquires the association ID of the image to be subjected to the image processing from the first image table 104, and generates a request message. The image that has already been uploaded can be subjected to the image processing. The request message is composed by a JavaScript (trademark) object notation (JSON) in the example illustrated in FIG. 6, but may be composed by an extensible markup language (XML) or another representation method.

The first job management unit 103 embeds the association ID acquired from the first image table 104 into the DataSetID field of the request message illustrated in FIG. 6. Specifically, the first job management unit 103 embeds the association IDs (latest image association ID and past image association ID) of two images being the generation sources of the subtraction image, into the DataSetID field. Further, the first job management unit 103 embeds an image processing type into the ActionCode field, and a processing priority into the ActionPriority field. The first job management unit 103 embeds an ID defined by the management apparatus 100 into the RequestSideID field. This ID is returned with its value being embedded as it is into the RequestSideID field of each of various messages of results of the image processing. The first job management unit 103 embeds one sentence for describing, for example, the location and the generation source of original data on the image to be processed, into the Description field. The first job management unit 103 embeds decryption information to be used when the image is encrypted and uploaded, into the DataSetDecryptionInfo field. The first job management unit 103 embeds an examination ID attached to an image created as the result of the image processing into the StudyInstanceUID field. The first job management unit 103 embeds a detail parameter for the image processing into the Params field.

Referring back to FIG. 5, after the processing of Step S500, in Step S501, the first communication processing unit 101 of the management apparatus 100 transmits the request message to the image processing apparatus 110. When the second communication processing unit 111 of the image processing apparatus 110 receives the request message in Step S501, the second job management unit 113 then issues, in Step S502, a job ID for managing image processing corresponding to the request message. Subsequently, in Step S503, the second image management unit 112 issues an association ID for an image (a subtraction image in the first embodiment) newly generated in response to the image processing relating to the request, and generates an image file name for the image. In Step S504, the second job management unit 113 stores the job ID, the association ID indicated in the request message, and the association ID of the newly generated image in the second job table 115 in association with one another. With this processing, for example, as illustrated in FIG. 7, the second job table 115 stores records (rows illustrated in FIG. 7) corresponding to two jobs. Each of the records includes the job ID, the association IDs (latest image association ID and past image association ID) of the images being the generation source of a subtraction image, and the association ID of the subtraction image.

Subsequently, in Step S505, the second image management unit 112 updates the second image table 114. Specifically, the second image management unit 112 stores the association ID of the newly generated subtraction image and a newly generated image file name corresponding to the subtraction image in the second image table 114 in association with each other. In this case, the type (subtraction) of the image processing is stored in association with the generation source. With this processing, as illustrated in FIG. 7, two records for the association IDs “7” and “8” are added to the second image table 114. At this time point, an image (subtraction image) corresponding to the image file name of the newly added record is not generated. Therefore, no image is associated with the image file name of the newly added record. Subsequently, in Step S506, the second communication processing unit 111 transmits to the management apparatus 100 the job ID issued in Step S502 and the association ID and image file name of the image generated in the image processing.

In Step S506, when the first communication processing unit 101 of the management apparatus 100 receives the job ID, the association ID, and the image file name, the CPU 202 of the management apparatus 100 advances the processing to Step S507. In Step S507, the first job management unit 103 of the management apparatus 100 identifies the association IDs (latest image association ID and past image association ID) included in the request message transmitted in Step S500. Then, the first job management unit 103 stores the job ID, the association ID of the image generated in the image processing, the latest image association ID, and the past image association ID in the first job table 105 in association with one another. In the first embodiment, the first job management unit 103 refers to the first image table 104 to store the patient name associated with the association ID in the first job table 105 further in association with the job ID. With this processing, as illustrated in FIG. 7, two records are added to the first job table 105.

In the first embodiment, the first communication processing unit 101 transmits, in Step S506, the association ID and image file name of the image generated in the image processing to the management apparatus 100 as a response to the request message, but may transmit the association ID and image file name after the image processing is completed. That is, the first communication processing unit 101 may transmit only the job ID as a response to the request message in Step S506, and after the image processing is completed, transmit the job ID and the associated ID and image file name of the generated image to the management apparatus 100. The image file name is not required to be transmitted when the image file name can be derived from the association ID based on a predetermined rule.

Subsequently, in Step S508, the first image management unit 102 updates the first image table 104. Specifically, the first image management unit 102 stores the association ID of the newly generated subtraction image and a newly generated image file name corresponding to the subtraction image in association with each other. In the first embodiment, the first job management unit 103 further identifies the personal information through use of the association IDs of the latest image and past image associated with the association ID of the subtraction image in the first job table 105 as search keys. Then, the first job management unit 103 stores the identified personal information in association with the association ID of the newly generated subtraction image. With this processing, as illustrated in FIG. 7, the two records for association IDs “7” and “8” are added to the first image table 104. At this time point, the main image data is not associated with the image file name newly added to the first image table 104. When an image corresponding to the image file name added to the first image table 104 and the second image table 114 is generated, the main image data is stored in each of the first image table 104 and the second image table 114 in association with the image file name. In this case, the main image data is stored by transmitting the main image data corresponding to the association ID from the second communication processing unit 111 of the image processing apparatus 110 to the first communication processing unit 101 of the management apparatus 100.

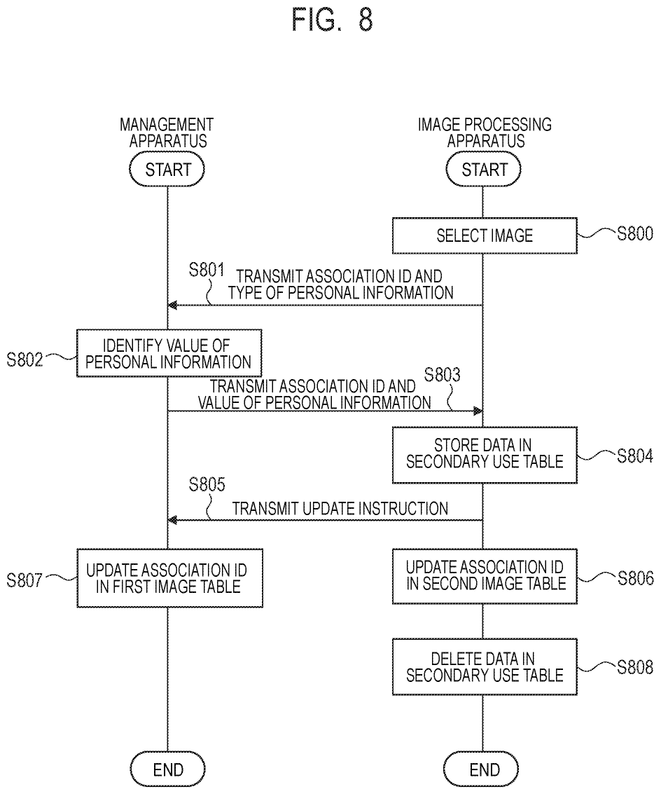

FIG. 8 is a sequence diagram for illustrating secondary use processing to be performed by the medical system. The image uploaded to the image processing apparatus 110 may be secondarily used. In this case, the image processing apparatus 110 creates secondary use data by adding required personal information to the image to be secondarily used. The secondary use processing is processing relating to creation of secondary use data. In this case, a case in which data obtained by associating an image generated by a CT apparatus with age information among pieces of personal information is secondarily used is described as an example. In this case, information in which the image and the age are associated with each other is generated as the secondary use data.

In Step S800, the secondary use management unit 117 of the image processing apparatus 110 selects the image to be secondarily used from the second image table 114. For example, when an image is designated as an image whose generation source is a CT apparatus among the images stored in the second image table 114 illustrated in FIG. 7, the secondary use management unit 117 selects four images of the association IDs “1”, “2”, “3”, and “4”. The secondary use management unit 117 sets, as an image to be selected, an image designated based on, for example, a user operation performed through the image processing apparatus 110 or a user operation performed through the external apparatus. Subsequently, in Step S801, the second communication processing unit 111 transmits, to the management apparatus 100, information including the association ID of the image selected in Step S800 and the type of the personal information required for the secondary use. In this case, the “age” is indicated as the type of the personal information.

When the first communication processing unit 101 of the management apparatus 100 receives the association ID and the type of the personal information in Step S801, the first image management unit 102 then identifies the value of the personal information in Step S802. Specifically, the first image management unit 102 refers to the first image table 104 to identify a value corresponding to the type of the personal information (value of the personal information) among the pieces of personal information associated with the association ID received in Step S801. In this case, the age is identified. Subsequently, in Step S803, the first communication processing unit 101 transmits, to the image processing apparatus 110, a combination of the association ID and the value corresponding to the type of the personal information identified in Step S802 (value of the age). In this case, four combinations of the association IDs “1” to “4” and the respective values of the age are transmitted to the image processing apparatus 110.

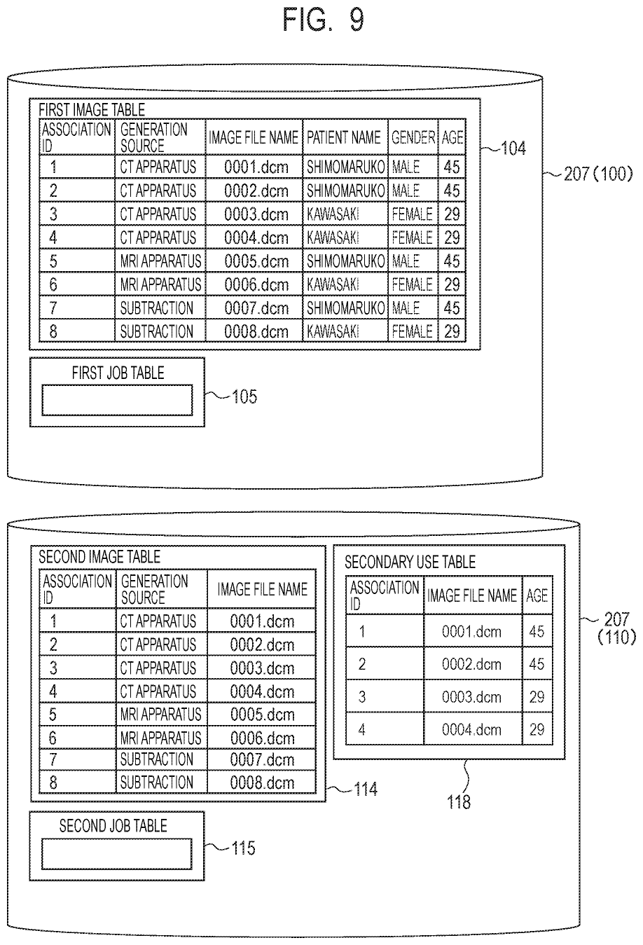

When the second communication processing unit 111 of the image processing apparatus 110 receives the combination of the association ID and the value of the personal information in Step S803, the CPU 202 of the image processing apparatus 110 advances the processing to Step S804. In Step S804, the secondary use management unit 117 of the image processing apparatus 110 stores, in the secondary use table 118, the combination of the association ID and the value of the personal information received in Step S803. With this processing, as illustrated in FIG. 9, records corresponding to the four respective association IDs are added to the secondary use table 118. In this case, in the case of simple statistical processing using only the age or other such secondary use that does not require the image itself, the secondary use table 118 is not required to store the image file name. At this time point, the job is finished, and all the records in the first job table 105 and the second job table 115 are deleted. This processing is an example of association processing for associating the personal information and the association ID being the identification information of the medical information with each other.

Subsequently, in Step S805, the second image management unit 112 determines that an update timing for the association ID has been reached, and uses the second communication processing unit 111 to transmit an update instruction for the association ID to the image management apparatus 100. When receiving the association ID and the value of the personal information, the second image management unit 112 determines that the update timing has been reached. As another example, when new data is stored in the secondary use table, the second image management unit 112 determines that the update timing has been reached. Subsequently, in Step S806, when determining that the update timing has been reached, the second image management unit 112 updates the association ID stored in the second image table 114 to a new association ID. In this case, the processing of Step S805 is an example of update control processing for controlling the start of the updating.

Meanwhile, when the first communication processing unit 101 of the management apparatus 100 receives the update instruction in Step S805, the first image management unit 102 of the management apparatus 100 then updates, in Step S807, the association ID stored in the first image table 104 to a new association ID. The second image table 114 and the first image table 104 store the same image, and the second image management unit 112 and the first image management unit 102 update, in the update processing, the association ID so that the same association ID is assigned to the same image. Specifically, it is assumed that the second image management unit 112 and the first image management unit 102 update the association ID to the same new association ID by applying the same injective function to the association ID.

It is also assumed that the processing of Step S806 and Step S807 are performed in synchronization with each other. For example, it is assumed that, through the designation of the update timing performed in the update instruction, the first image management unit 102 and the second image management unit 112 perform update processing for the association ID at the same timing or at timings within a predetermined time range, or other such corresponding timings. In addition, the first image management unit 102 and the second image management unit 112 perform control so as to inhibit various operations and various kinds of processing from being performed on the image associated with the association ID to be updated, in order to prevent a conflict from occurring in image management information during the update processing for the association ID.

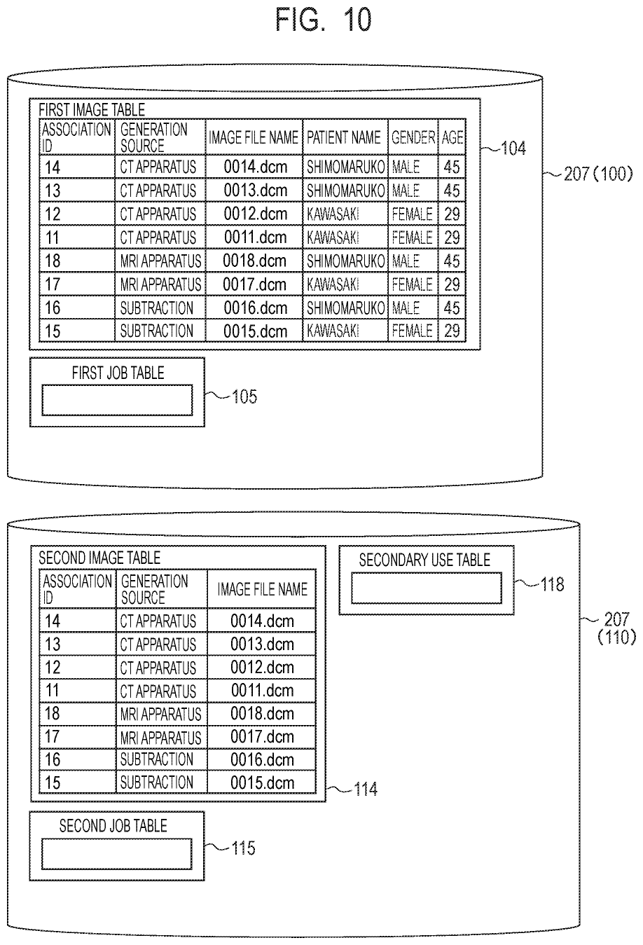

In addition, in the image processing apparatus 110, after the processing of Step S806, the secondary use management unit 117 deletes all pieces of data (records) in the secondary use table 118 in Step S808. The processing of Step S808 is only required to be performed after the end of the secondary use, and is not necessarily performed after the processing of Step S806. FIG. 10 is a diagram for illustrating an example of data structures of the data storage areas 207 at the end time point of the secondary use processing. In this manner, all pieces of data in the secondary use table 118 are deleted. Meanwhile, all the association IDs in the first image table 104 and the second image table 114 are updated to new association IDs. In the first embodiment, the image file names are also updated when the association IDs of the first image table 104 and the second image table 114 are updated, but the present disclosure is not limited thereto, and the file names may not be updated when the association IDs are updated.

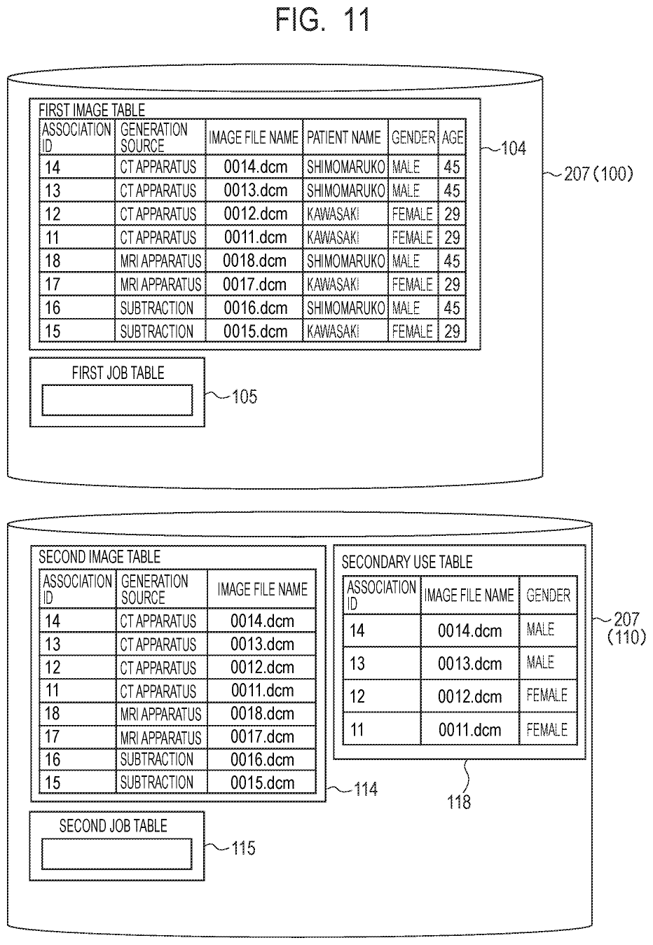

In this manner, in the first embodiment, immediately after the secondary use data is created in the image processing apparatus 110, the association IDs associated with the images are updated in the first image table 104 and the second image table 114. With this processing, next time secondary use data is generated, the association ID is already updated. Therefore, even when secondary use data generated at a first time point and secondary use data generated at a second time point after the first time point are pieces of data that correspond to the same image, the two pieces of secondary use data include different association IDs. For example, the association IDs stored in the secondary use table are “1”, “2”, “3”, and “4” in the example of FIG. 9, while the association IDs stored in the secondary use table are “14”, “13”, “12”, and “11” in the example of FIG. 11. Therefore, for example, even when the secondary use data in the secondary use table 118 is continuously leaked, the unfavorable identification of the patient or other such problem due to the information leakage can be prevented by accumulating the personal information with the association ID being used as a key.

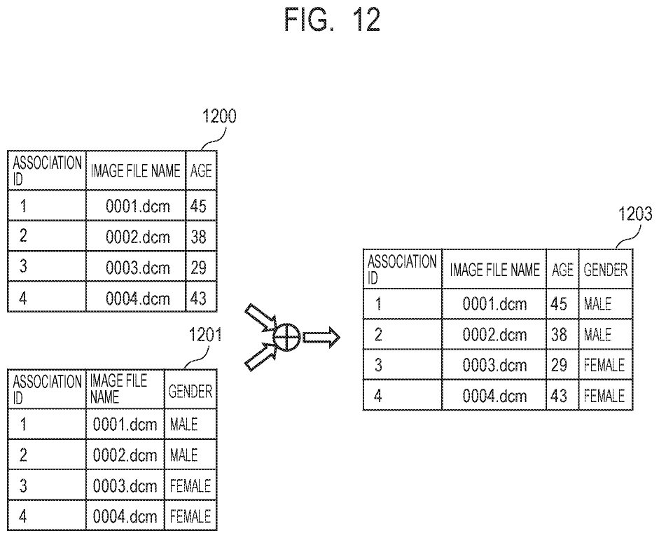

When the association ID is not to be updated, for example, as illustrated in FIG. 12, a piece of secondary use data 1200 is generated at the first time point, and a piece of secondary use data 1201 is then generated at the second time point. In this case, when the two pieces of secondary use data 1200 and 1201 are leaked, the age and the gender can be accumulated as the personal information of the person corresponding to the association ID as indicated by data 1203. This increases the number of pieces of information for identifying an individual in such a manner that, for example, a person corresponding to the association ID “1” is a 45-year-old male.

Meanwhile, as described above, the association ID is updated by the medical system in the first embodiment, to thereby be able to prevent an individual from being identified based on the accumulation of the personal information associated with the association ID. In addition, a correspondence relationship between the association IDs associated with the same image in the management apparatus and the image processing apparatus can be maintained before and after the update, and hence the identity of the images can be determined in both apparatus. As described above, the medical system in the first embodiment can provide a mechanism that allows the secondary use of a medical image of a patient while protecting the personal information of the patient.

As a first modification example of the first embodiment, the image processing apparatus 110 and the management apparatus 100 may be configured to periodically update the association ID even after the association ID is synchronously updated in Step S806 and Step S807. In this case, the image processing apparatus 110 and the management apparatus 100 each use, for example, a task scheduler to synchronously update the association ID.

As a second modification example of the first embodiment, an update interval in the periodic update of the association ID may be determined depending on the type of the personal information to be secondarily used. For example, when the type of the personal information is the gender, an individual identification level can be considered to be low due to the existence of only two types of male and female. In view of this, the image processing apparatus 110 determines the update interval depending on the level of the possibility that the individual can be identified by the personal information. When the type of the personal information is the gender, the update interval is set to a relatively long period. Meanwhile, when applicable patients are from 0 years old to 99 years old, the age can take 100 different values, which can be considered to be high in individual identification level. In view of this, the image processing apparatus 110 determines the update interval depending on the level of the possibility that the individual can be identified by the personal information. When the type of the personal information is the age, a shorter period than that of the gender is determined as the update interval. Processing for determining the update interval may be performed by any one of the management apparatus 100 and the image processing apparatus 110.

As a third modification example of the first embodiment, the medical system may be configured to periodically update the association ID irrespective of the generation timing of new secondary use data. Even in this case, by appropriately setting the update interval, the association ID can be updated at the timing during a period to elapse after a piece of secondary use data is generated until the next piece of secondary use data is created.

As a fourth modification example of the first embodiment, the apparatus configured to control the update of the association ID, that is, the apparatus configured to determine the update timing of the association ID may be any one of the management apparatus 100 and the image processing apparatus 110. The management apparatus 100 may determine whether or not the update timing has been reached, and transmit the update instruction to the image processing apparatus 110. As another example, an external apparatus other than the management apparatus 100 and the image processing apparatus 110 may determine whether or not the update timing has been reached, and transmit the update instruction to the management apparatus 100 and the image processing apparatus 110. For example, the external apparatus may periodically transmit the update instruction as described above. As still another example, the external apparatus may monitor the image processing apparatus 110, and transmit the update instruction to the image processing apparatus 110 and the management apparatus 100 when the secondary use table is created.

As a fifth modification example of the first embodiment, the processing (Step S805 to Step S807 illustrated in FIG. 8) for updating the same association ID associated with the same image stored in the first image table 104 and the second image table 114 to the same new association ID is not limited to the processing in the first embodiment. As another example, the management apparatus 100 may issue a new association ID, and the management apparatus 100 and the image processing apparatus 110 may update the association ID through use of the new association ID issued by the management apparatus 100. Specifically, the first image management unit 102 of the management apparatus 100 issues a new association ID for the image that has been transmitted to the image processing apparatus 110. Then, the first image management unit 102 generates update information in which the newly issued association ID and the old association ID that has been associated so far are associated with each other. The update information is transmitted to the image processing apparatus 110 by the first communication processing unit 101. The first image management unit 102 updates the association ID stored in the first image table 104 to the newly issued association ID, and the second image management unit 112 of the image processing apparatus 110 updates the association ID of the second image table 114 based on the update information. Even in this case, the update of the first image table 104 and the update of the second image table 114 are assumed to be performed in synchronization with each other. In contrast, the image processing apparatus 110 may issue a new association ID for the image stored in the second image table 114, and the association ID in the first image table 104 and the second image table 114 may be updated based on the newly issued association ID.

A sixth modification example of the first embodiment is described. In the first embodiment, the same association ID is assumed to be associated with the same image in the first image table 104 and the second image table 114 both before and after the update. However, in the first image table 104 and the second image table 114, the association IDs associated with the same image are only required to be information that allows the management apparatus 100 and the image processing apparatus 110 to identify that both images are the same image. That is, the association IDs associated with the same image are not limited to the same information. For example, the association IDs associated with the same image may be five-digit numbers with the lower three digits having the same value and the remaining two digits having different values (any values). As another example, the association IDs associated with the same image may be values obtained before and after the association ID is converted by a predetermined function.

As a seventh modification example of the first embodiment, the exemplary case in which a medical image is to be processed is described in the first embodiment, but information to be processed may be any kind of medical information obtained for a given person, and is not limited to the medical image. Examples of the medical information other than the medical image include an electrocardiogram and examination data. In this case, an apparatus to which the medical information is to be transmitted is an information processing apparatus configured to perform specific kind of processing on the medical information in place of the image processing apparatus.

### Second Embodiment

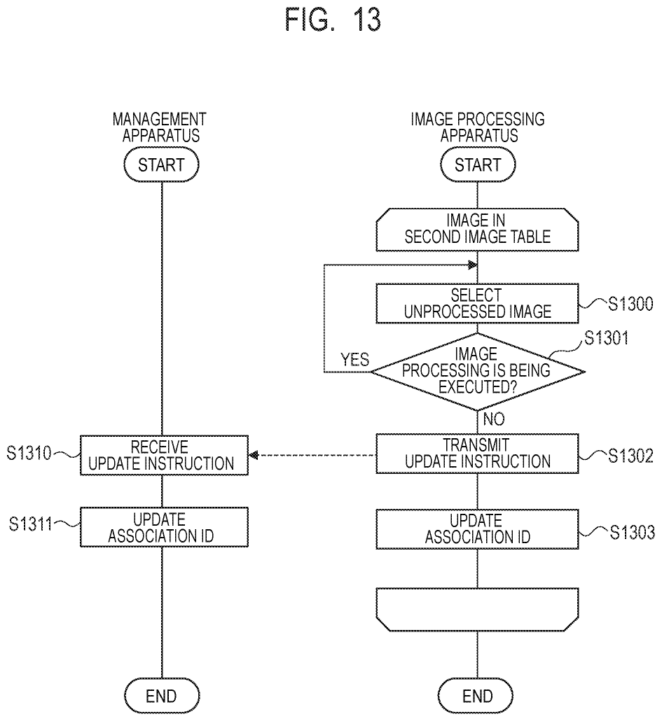

A medical system in a second embodiment of the present disclosure is configured to perform control so as to avoid updating an association ID while image processing using an image corresponding to the association ID to be updated is being performed. The medical system in the second embodiment is described below in terms of differences from the medical system in the first embodiment. FIG. 13 is a flow chart for illustrating the update control processing for the association ID to be performed by the medical system in the second embodiment. This processing is processing corresponding to Step S805 to Step S807 described with reference to FIG. 8 in the first embodiment.

The second image management unit 112 of the image processing apparatus 110 performs the processing from Step S1300 to Step S1303 on all the images stored in the second image table 114. That is, in Step S1300, the second image management unit 112 selects, from among the images stored in the second image table 114, an image whose association ID has not been updated. Subsequently, in Step S1301, the second image management unit 112 examines whether or not image processing using the selected image is being executed. When the image processing is not being executed (No in Step S1301), the second image management unit 112 advances the processing to Step S1302. When the image processing is being executed (Yes in Step S1301), the second image management unit 112 returns the processing to Step S1300 to change the image to be processed.

In Step S1302, the second communication processing unit 111 transmits an update instruction for the association ID to the image management apparatus 100. Subsequently, in Step S1303, the second image management unit 112 updates, in the second image table 114, the association ID of the image to be updated. Meanwhile, in Step S1310, the first communication processing unit 101 of the management apparatus 100 receives the update instruction. Subsequently, in Step S1311, the first image management unit 102 updates, in the first image table 104, the association ID to be updated. The update processing of Step S1303 and the update processing of Step S1311 are assumed to be executed at corresponding timings.

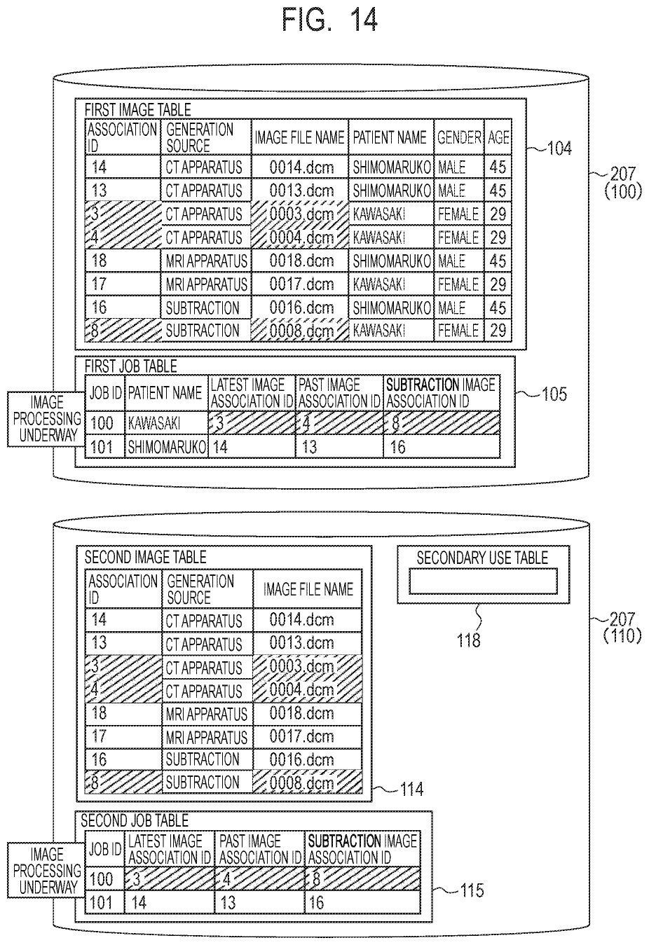

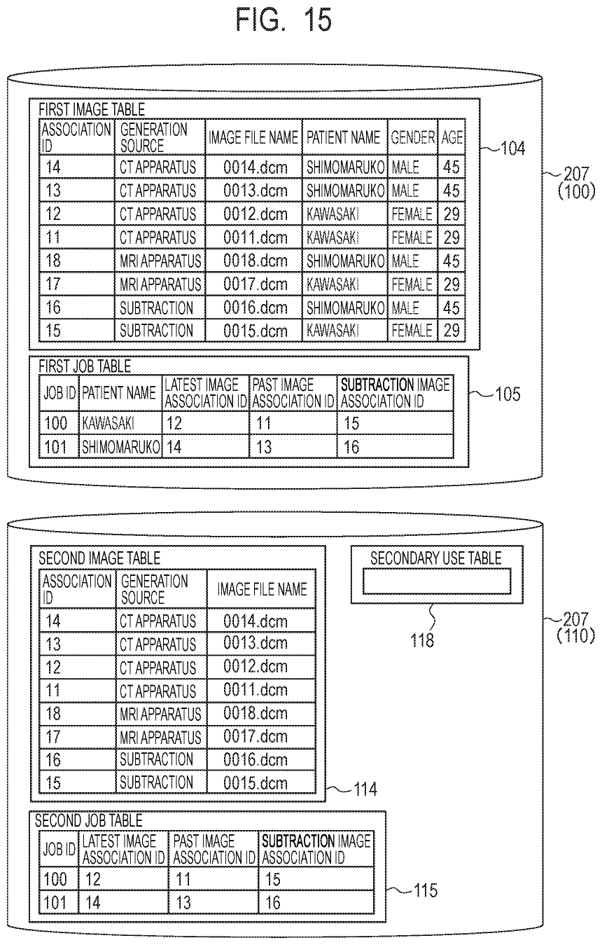

With this processing, it is assumed that, for example, the image processing of the job ID “100” is being executed under the state illustrated in FIG. 7. In this case, when the update control processing is executed, the association IDs other than the association IDs relating to the image processing being executed are updated as the association ID as illustrated in FIG. 14. In the example of FIG. 14, the association IDs “3”, “4”, and “8” relating to the image processing of the job ID “100” are not updated. Meanwhile, the association IDs other than those association IDs are updated. After that, when the image processing of the job ID “100” is brought to an end, as illustrated in FIG. 15, the association IDs “3”, “4”, and “8” relating to the image processing of the job ID are also subjected to the update processing to be updated to new association IDs. The other configuration and processing of the medical system in the second embodiment are the same as the configuration and processing of the medical system in the first embodiment.

In the second embodiment, as described above, the relevant association IDs are not updated during the image processing. This can suppress an occurrence of an error. There is also a case in which a plurality of information processing apparatus may cooperate to perform the image processing in a distributed manner through use of the association ID. In this case, it is difficult to propagate the change of the association ID to a large number of information processing apparatus. In contrast, in the second embodiment, it is possible to simply configure a cooperative system by avoiding updating the relevant association ID during the image processing.

### Third Embodiment

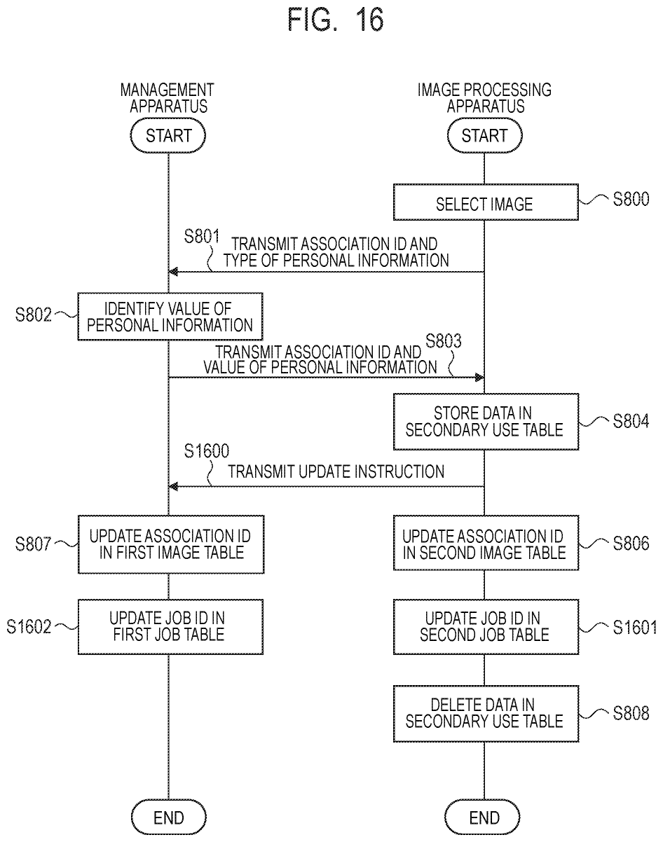

The medical system in a third embodiment of the present disclosure updates job IDs in the second job table 115 and the first job table 105 as well when the association IDs are updated. The medical system in the third embodiment is described below in terms of differences from the medical system in the first embodiment. FIG. 16 is a sequence diagram for illustrating secondary use processing to be performed by the medical system in the third embodiment. Of the processing steps in the secondary use processing illustrated in FIG. 16, the same processing steps as those of the secondary use processing in the first embodiment described with reference to FIG. 8 are denoted by the same reference symbols.

After the processing of Step S804, in Step S1600, the second communication processing unit 111 of the image processing apparatus 110 transmits an update instruction for the association ID and the job ID. Subsequently, in Step S806 and Step S807, the update processing for the association ID is performed by the second image management unit 112 and the first image management unit 102 in synchronization with each other. After that, in Step S1601, the second job management unit 113 updates the job ID stored in the second job table 115 to a new job ID. In the same manner, in Step S1602, the first job management unit 103 updates the job ID stored in the first job table 105 to a new job ID. The job ID in the second job table 115 and the job ID in the first job table 105 are updated so that the same new job ID is assigned to the same job at the corresponding timings. The processing for updating the job ID is the same as the processing for updating the association ID. The processing order of the update of the association ID and the update of the job ID is not limited to the processing order in the third embodiment, and both kinds of processing may be simultaneously performed, or the job ID may be updated first.

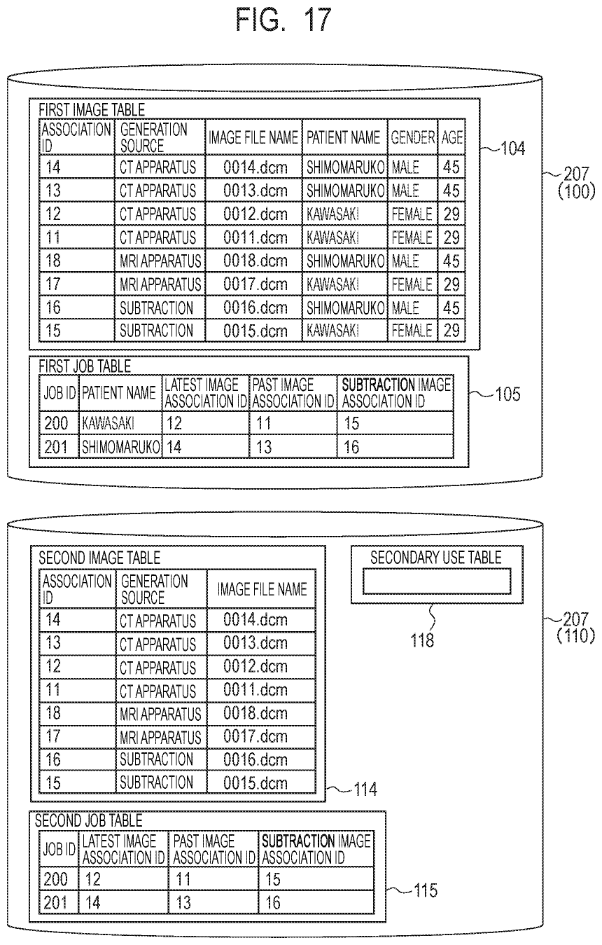

FIG. 17 is a diagram for illustrating a case in which both the association ID and the job ID are updated under the state illustrated in FIG. 7. In the example of FIG. 17, the job IDs “100” and “101” are updated to the job IDs “200” and “201”, respectively. The other configuration and processing of the medical system in the third embodiment are the same as the configuration and processing of the medical system in each of the other embodiments.

When the information in the second job table 115 is continuously leaked, it is possible to track the update of the association ID by associating the association ID with the job ID in the second job table 115. Meanwhile, by also updating the job ID as described in the third embodiment, it is possible to prevent the update of the association ID from being tracked.

According to each of the embodiments described above, it is possible to provide the mechanism capable of secondary use of a medical image of a patient while protecting the personal information of the patient.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

Specifically, the present disclosure may be applied to a system formed of a plurality of devices (for example, a host computer, an interface device, an image pickup apparatus, and a Web application), or may be applied to an apparatus formed of a single device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions

What is claimed is:

1. An information processing system comprising:
a first information processing apparatus; and
a second information processing apparatus,
the first information processing apparatus including:
a first storage unit configured to store personal information of a patient, medical information obtained for a person relating to the personal information, and identification information for identifying the medical information in association with one another; and
a first update unit configured to update the identification information stored in the first storage unit,
the second information processing apparatus including:
a second storage unit configured to store the medical information and the identification information in association with each other;
a reception unit configured to receive at least a part of the personal information associated with one piece of identification information from the first information processing apparatus;
an associating unit configured to associate the personal information received by the reception unit with the one piece of identification information; and
a second update unit configured to update the identification information stored in the second storage unit, and
the first update unit and the second update unit configured to update a first piece of identification information associated with the same medical information in the first storage unit and the second storage unit, respectively at corresponding timings, to a second piece of identification information different from the first piece of identification information.

2. The information processing system according to claim 1, wherein the first information processing apparatus further includes an update control unit configured to control a start of the updating to be performed by the first update unit and the second update unit.

3. The information processing system according to claim 1, wherein the second information processing apparatus further includes an update control unit configured to control a start of the updating to be performed by the first update unit and the second update unit.

4. The information processing system according to claim 3, wherein the update control unit is configured to control the updating to be started, when the reception unit receives the personal information.

5. The information processing system according to claim 3, wherein the update control unit is configured to control the updating to be started periodically.

6. The information processing system according to claim 5, wherein the update control unit is configured to perform the updating at an interval corresponding to a type of the personal information received by the reception unit.

7. The information processing system according to claim 1, wherein the first update unit and the second update unit are configured to update the first piece of identification information to the second piece of identification information through application of the same injective function.

8. The information processing system according to claim 3,
wherein the second information processing apparatus further includes a processing unit configured to perform processing that uses the medical information, and
wherein the update control unit is configured to perform control so that the identification information of the medical information being used in the processing performed by the processing unit is inhibited from being updated.

9. The information processing system according to claim 8,
wherein the second information processing apparatus further includes a third storage unit configured to store identification information of medical information used in the processing performed by the processing unit in association with identification information for identifying the processing, and
wherein the second update unit is configured to update the identification information of the processing stored in the third storage unit to another piece of identification information, when the medical information is updated.

10. The information processing system according to claim 1,
wherein the reception unit of the second information processing apparatus is configured to receive the medical information from the first information processing apparatus,
wherein the second information processing apparatus further includes:
an issuance unit configured to issue the identification information for the medical information received by the reception unit;
a management unit configured to store, in the second storage unit, the medical information received by the reception unit and the identification information issued for the medical information in association with each other; and
a transmission unit configured to transmit the identification information to the first information processing apparatus, and
wherein the first information processing apparatus further includes a management unit configured to store, in the first storage unit, the medical information transmitted to the second information processing apparatus and the identification information transmitted from the second information processing apparatus in association with each other.

11. The information processing system according to claim 1,
wherein the medical information comprises a medical image, and
wherein the second information processing apparatus further includes an image processing unit configured to perform image processing on the medical image.

12. An information processing apparatus comprising:
a first storage unit configured to store personal information, medical information obtained for a person relating to the personal information, and identification information for identifying the medical information in association with one another; and
an update unit configured to update the identification information stored in the first storage unit,
the update unit configured to update a first piece of identification information associated with the same medical information in a second storage unit of an external apparatus, which is configured to store the medical information and the identification information in association with each other, and the first storage unit, respectively at corresponding timings, to a second piece of identification information different from the first piece of identification information.

13. An information processing apparatus comprising:
a first storage unit configured to store medical information and identification information for identifying the medical information in association with each other;
a reception unit configured to receive at least a part of personal information associated with one piece of identification information from an external apparatus including a second storage unit configured to store personal information, the medical information obtained for a person relating to the personal information, and the identification information in association with one another;
an associating unit configured to associate the personal information received by the reception unit with the one piece of identification information; and
an update unit configured to update the identification information stored in the first storage unit,
the update unit configured to update a first piece of identification information associated with the same medical information in the first storage unit and the second storage unit, respectively at corresponding timings, to a second piece of identification information different from the first piece of identification information.
